(19) United States
(12) Patent Application Publication
(10) Pub. No.: US 2013/0012916 A1
(43) Pub. Date: Jan. 10, 2013

(54) DELIVERY OF IMMUNOGLOBULIN VARIABLE DOMAINS AND CONSTRUCTS THEREOF

(75) Inventors: Christine Labeur, Brugge (BE); Hilde Adi Pierrette Revets, Meise (BE); Nasir Hussain, Harrow (GB); Charles Potter, Standlake (GB)

(73) Assignees: Glide Pharmaceutical Technologies Limited, Abingdon (GB); Ablynx N.V., Zwijnaarde (BE)

(21) Appl. No.: 13/577,947

(22) PCT Filed: Feb. 10, 2011
(86) PCT No.: PCT/EP2011/051955

§ 371 Date: Sep. 26, 2012

Related U.S. Application Data
(60) Provisional application No. 61/303,451, filed on Feb. 11, 2010.

(51) Int. Cl. A61K 39/395; A61M 5/30; A61K 9/00

(52) U.S. Cl. 604/500; 424/400; 424/136.1; 424/130.1; 604/68

(57) ABSTRACT

The present invention relates to formulations and methods for administering therapeutic molecules comprising immunoglobulin variable domains using needle-free delivery devices.

### FIELD OF THE INVENTION

The field of invention relates to formulation and administration of immunoglobulin variable domains and constructs thereof.

### BACKGROUND OF THE INVENTION

Medicine injector pens have been developed to permit a patient to self-administer proper doses of medicine that can not be administered orally. While injector pens are widely used, their use can be challenging for patients with sensitive or damaged skin, for patients with needle-phobia, and for very young patients. In addition, injector pens carry the risk of needle-stick injuries through inadvertent use, which can result in cross-contamination. To reduce the problems associated with the injector pens, needle-free delivery devices for self-administration have been developed. Needle-free delivery devices can administer medicine or diagnostics through the skin such as by means of high pressure, electric current or ultrasound.

Unless explicitly mentioned and/or defined otherwise herein, all terms mentioned herein have the meaning given in WO 2008/020079. For example, the term “Nanobody” is as defined in WO 2008/020079, and thus in a specific aspect generally denotes a VHH, a humanized VHH or a camelized VH (such as a camelized human VH) or generally a sequence optimized VHH (such as e.g. optimized for chemical stability and/or solubility, maximum overlap with known human framework regions and maximum expression). Also, when referring to a construct, compound, protein or polypeptide of the invention, terms like “monovalent”, “bivalent” (or “multivalent”), “bispecific” (or “multispecific”), and “biparatopic” (or “multiparatopic”) have the meaning given in WO 2008/020079.

When a term is not specifically defined, it has its usual meaning in the art, which will be clear to the skilled person. Reference is for example made to the standard handbooks, such as Sambrook et al, “Molecular Cloning: A Laboratory Manual” (2nd.Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10th Ed. Blackwell Publishing, UK (2001); and Janeway et al., “Immunobiology” (6th Ed.), Garland Science Publishing/Churchill Livingstone, New York (2005), as well as to the general background art cited herein.

### SUMMARY OF THE INVENTION

Immunoglobulin variable domains can bind antigens and have superior therapeutic properties, including stability and bio-availability, when compared to traditional antibodies. New formulations and methods of administration of immunoglobulin variable domain using needle-free delivery devices are desired.

In certain aspects, the invention provides formulations, devices and methods for the administration of therapeutic molecules that include one or more immunoglobulin variable domains, or other protein scaffolds, and constructs thereof.

In one aspect, formulations including one or more therapeutic molecules and one or more excipients are provided. The formulations include at least 80% by mass of the one or more therapeutic molecules, and the one or more therapeutic molecules include one or more immunoglobulin variable domains.

In some embodiments, the formulation includes at least 90% by mass of the one or more therapeutic molecules. In some of the foregoing embodiments, the formulation is a solid. In some of the foregoing embodiments, the formulation is shaped for delivery to a subject through the subject's skin. In some of the foregoing embodiments, the formulation is a rod formulation.

In some of the foregoing embodiments, the solubility of the one or more therapeutic molecules at a concentration of at least 50 mg/ml in a physiological solution is greater than 99%. In some of the foregoing embodiments, the solubility of the one or more therapeutic molecules at a concentration of at least 100 mg/ml in a physiological solution is greater than 99%. In some of the foregoing embodiments, the physiological solution is isotonic and has a neutral pH.

In some of the foregoing embodiments, the formulation is at least about 1 milligram in total mass. In some of the foregoing embodiments, the formulation is at least about 3 milligrams in total mass. In some of the foregoing embodiments, the formulation is at least about 10 milligrams in total mass. In some of the foregoing embodiments, the formulation is at least about 14 milligrams in total mass.

In some of the foregoing embodiments, administration of the formulation to a subject results in the delivery to the subject of at least 0.8 mg of the one or more therapeutic molecules per administration event. In some of the foregoing embodiments, the formulation is constructed and arranged to deliver to a subject at least 0.8 mg of the one or more therapeutic molecules when administered to a subject.

In some of the foregoing embodiments, administration of the formulation to a subject results in the delivery to the subject of at least 2.4 mg of the one or more therapeutic molecules per administration event. In some of the foregoing embodiments, the formulation is constructed and arranged to deliver to a subject at least 2.4 mg of the one or more therapeutic molecules when administered to a subject.

In some of the foregoing embodiments, administration of the formulation to a subject results in the delivery to the subject of at least 8 mg of the one or more therapeutic molecules per administration event. In some of the foregoing embodiments, the formulation is constructed and arranged to deliver to a subject at least 8 mg of the one or more therapeutic molecules when administered to a subject.

An immunoglobulin variable domain is generally defined herein as an amino acid sequence that comprises an immunoglobulin fold or may be an amino acid sequence that, under suitable conditions (such as physiological conditions) is capable of forming an immunoglobulin fold (i.e. by folding). Reference is inter alia made to the review by Halaby et al., J. (1999) Protein Eng. 12, 563-71. As will be known to the skilled person, an immunoglobulin variable domain essentially consists of four framework regions and three CDR's.

In some of the foregoing embodiments, the immunoglobulin variable domain is a VH, VL, VHH, camelized VH, camelized VL, or VHH that is optimized for stability, potency, manufacturability and similarity to human framework regions. For example, the immunoglobulin domain may be a Nanobody (e.g. a VHH, a humanized VHH or a camelized VH, such as a camelized human VH).

The invention is particularly suitable for immunoglobulin variable domains that can functionally bind to an antigen without the presence of, and without any interaction with another variable domain sequence (such as a VH/VL interaction). Again, preferred but some non-limiting examples are Nanobodies (e.g. a VHH, a humanized VHH or a humanized VH, such as a humanized human VH), dAb's, domain antibodies, single domain antibodies and other immunoglobulin single variable domains.

In some of the foregoing embodiments, the immunoglobulin variable domain is a VHH that is optimized for stability, potency, manufacturability and similarity to human framework regions.

In some of the foregoing embodiments, the therapeutic molecule comprises a multivalent and/or multispecific construct.

In another aspect, needle-free delivery devices are provided. The needle-free delivery devices including the formulation of any of the foregoing embodiments.

In some of the foregoing embodiments, the needle-free delivery device includes: i) a housing; ii) a means for generating a force capable of pushing the formulation from a packaging into a human or animal body; iii) a means for transmitting said force to push the formulation from the packaging into the human or animal body; and iv) a means for triggering the device.

In some of the foregoing embodiments, the needle-free delivery device includes: i) a housing; ii) a way of generating a force capable of pushing the formulation from a packaging into a human or animal body; iii) a way of transmitting said force to push the formulation from the packaging into the human or animal body; and iv) a way of triggering the device.

In some of the foregoing embodiments, the needle-free delivery device includes: i) a housing; ii) a force generator configured to generate a force capable of pushing the formulation from a packaging into a human or animal body; iii) a force transmitter configured to transmit said force to push the formulation from the packaging into the human or animal body; and iv) a triggering element configured to trigger the device.

In still another aspect, methods for administering formulations to a subject are provided. The methods include administering to the subject the formulation of any of any of the foregoing embodiments by using a needle free delivery device.

In some of the foregoing embodiments, the needle-free delivery device includes: i) a housing; ii) a means for generating a force capable of pushing the formulation from a packaging into a human or animal body; iii) a means for transmitting said force to push the formulation from the packaging into the human or animal body; and iv) a means for triggering the device.

In some of the foregoing embodiments, the needle-free delivery device includes: i) a housing; ii) a way of generating a force capable of pushing the formulation from a packaging into a human or animal body; iii) a way of transmitting said force to push the formulation from the packaging into the human or animal body; and iv) a way of triggering the device.

In some of the foregoing embodiments, the needle-free delivery device includes: i) a housing; ii) a force generator configured to generate a force capable of pushing the formulation from a packaging into a human or animal body; iii) a force transmitter configured to transmit said force to push the formulation from the packaging into the human or animal body; and iv) a triggering element configured to trigger the device.

In some of the foregoing embodiments, the one or more therapeutic molecules has at least about 90% of the potency after formulation as prior to formulation. In some of the foregoing embodiments, the one or more therapeutic molecules has at least about 90% of the potency after administration as prior to administration. In some of the foregoing embodiments, the one or more therapeutic molecules has at least about 90% of the potency after administration as prior to formulation. In some of the foregoing embodiments, the potency is at least about 95%. In some of the foregoing embodiments, the potency is at least about 99%.

These and other aspects and embodiments of the invention are described in greater detail below.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 shows SEC chromatograms of 25 μg lyophilised vWF0002 resuspended in saline and the control sample (before lyophilisation) (λ=280 nm).

FIG. 2 shows RPC chromatograms of 25 μg lyophilised vWF0002 resuspended in saline and the control sample (before lyophilisation) (λ=280 nm).

FIG. 3 shows SEC chromatograms of 25 μg lyophilised vWF0002 resuspended in saline and the different vWF0002 formulations (90/10 rod carboxymethylcellulose, 80/20 Dextran, 80/20 methylcellulose) after resuspension.

FIG. 4 shows RPC chromatograms of 25 μg lyophilised vWF0002 resuspended in saline and the different vWF0002 formulations (90/10 rod carboxymethylcellulose, 80/20 Dextran, 80/20 methylcellulose) after resuspension.

FIG. 5 shows plasma concentration-time profiles for vWF0002 after subcutaneous implantation of a 3 mg 80/20 MC solid dosage formulation at a dose level of 80 μg/kg body weight and after a single subcutaneous administration at a dose level of 80 μg/kg body weight.

FIG. 6 shows mean % RICO-time profiles for vWF0002 after subcutaneous implantation of a 3 mg 80/20 MC solid dosage formulation at a dose level of 80 μg/kg body weight and after a single subcutaneous administration at a dose level of 80 μg/kg body weight.

FIG. 7 shows a plasma concentration-time profile for vWF0002 after subcutaneous implantation of a 10 mg 80/20 MC solid dosage formulation at a dose level of 276 μg/kg body weight.

FIG. 8 shows mean % RICO-time profile for vWF0002 after subcutaneous implantation of a 10 mg 80/20 MC solid dosage formulation at a dose level of 276 μg/kg body weight.

FIG. 9 shows a scheme for a lyophilization cycle.

### DETAILED DESCRIPTION OF THE INVENTION

In one aspect the invention provides methods, formulations and devices for the needle free delivery of therapeutic molecules comprising immunoglobulin variable domains, and therapeutic molecules based on other protein scaffolds. In some embodiments the formulations are administered by using a needle-free delivery device. In some embodiments, the needle-free delivery device is a GLIDE injector (as herein further defined). The delivery of therapeutic molecules comprising immunoglobulin variable domains by needle-free delivery devices provides certain benefits, including ease of use, reduced risk of contamination and accurate dosing, over other administration devices and methods.

### Formulations

In one aspect the invention provides formulations useful for the delivery of therapeutic molecules. In some embodiments the formulation comprises one or more therapeutic molecules and one or more excipients, wherein the therapeutic molecule comprises one or more immunoglobulin variable domains or another protein scaffold, and wherein the formulation comprises at least 80% by mass of the one or more therapeutic molecules. In some embodiments the formulation comprises at least 90% by mass of the one or more therapeutic molecules. In some embodiments the formulation is a solid.

In one aspect the invention provides solid formulations for the administration of therapeutic molecules comprising immunoglobulin variable domain(s). The solid formulations allow for the transdermal delivery of the therapeutic molecules comprising immunoglobulin variable domain(s). The solid formulations can be transdermally delivered, for instance by implanting of the solid formulations under the skin, by administering using a needle-free device, or by injection when prepared as a suspension. In some embodiments the solid formulations allow for the delivery of the therapeutic molecules by a needle-free device. In some embodiments the solid formulation comprises one or more excipients. Excipients provide the physical environment for the delivery of the therapeutic molecules as a solid formulation. It was surprisingly found, as described herein, that immunoglobulin variable domain-based therapeutic molecules could be formulated at a high percentage of the total mass of the formulation, while retaining solubility, and potency (such as pharmacodynamic and pharmacokinetic properties) of the unformulated therapeutic molecule.

Thus the invention provides for formulations with higher percentages of therapeutic molecules comprising immunoglobulin variable domains, and therapeutic molecules based on other protein scaffolds than previously described. The invention also accordingly provides for formulations with lower percentages of excipient than previously described. Thus, the methods of the invention allow for the delivery of more therapeutic molecules by mass in a formulation. In some embodiments, the formulation comprises at least 80% by mass of the one or more therapeutic molecules. In some embodiments the formulation comprises at least 90% by mass of the one or more therapeutic molecules. In some embodiments, the formulation comprises at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or up to 99.99% of therapeutic molecules by mass, preferably at least 80% of therapeutic molecules by mass, more preferably at least 90% of therapeutic molecules by mass.

The solid formulations comprise one or multiple excipients. The excipients used in the formulation can have a variety of functions, including filler, glidant, stabilizer and disintegrant. Suitable excipients are, in particular, fillers such as dextran or sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, anti/or polyvinylpyrrolidone (PVP).

If desired, disintegrating agents may be added, such as the cross-linked polyvinyl pyrrolidone, agar, or alginic acid or a salt thereof such as sodium alginate. Disintegrants include but are not limited to starch, including the commercial disintegrant based on starch, Explotab. Sodium starch glycolate, Amberlite, sodium carboxymethylcellulose, ultramylopectin, sodium alginate, gelatin, acid carboxymethyl cellulose, and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic molecule together to form a hard solid formulation and include materials from natural products such as acacia, tragacanth, starch and gelatin. Others include methyl cellulose (MC), ethyl cellulose (EC) and carboxymethyl cellulose (CMC).

An anti-frictional agent may be included in the formulation of the therapeutic to prevent sticking during the formulation process. Lubricants may be used as a layer between the therapeutic and an extruder wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that improve the flow properties of the drug during formulation and to aid rearrangement during extrusion may be added. The glidants may include starch, talc, pyrogenic silica and hydrated silicoaluminate.

Optionally, the formulation may also contain suitable stabilizers or agents which increase the solubility of the compounds to allow for the preparation of highly concentrated solutions.

The solid formulations described herein can have a variety of weights. For instance, the solid formulations used according to methods of the invention can be at least about 1 milligram in total mass, at least about 3 milligrams in total mass, at least about 10 milligrams in total mass or at least about 14 milligrams in total mass. In some embodiments, the formulations are at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 milligram or more in total mass. Because the formulations described herein allow for a high mass percentage of therapeutic molecules (e.g. 80% or 90% or higher of the total mass of the formulation), the solid formulations provide, for example, for the delivery of at least 0.8, at least 0.9, at least 1, at least 1.6, at least 1.8, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 milligrams or more of therapeutic molecules.

The solid formulations described can be in any shape of form. In general, the shape of the solid formulation will depend on the desired method of delivery. For instance, the solid formulations may be in a rod shape for delivery by a needle-free device or implantation under the skin. In some embodiments the formulation is a particle or powdered formulation that allows for the administration as a suspension.

In some embodiments the formulation is a rod formulation. A rod formulation as used herein is a formulation that is in the shape of a rod. A rod shape, as used herein, is a generally cylindrical shape having two ends, in which at least one of the ends is shaped to be sharp or pointed. In some embodiments the rod formulations allow for the delivery of the formulation by using a Glide Solid Dose Injector (Glide SDI®).

The Glide SDI pushes a water soluble, lipid soluble or otherwise biodegradable formulation through the skin of a human or animal. The formulation takes the form of a rod with a point on one end. Preferably the rod has a circular cross-section with a diameter of less than 3 mm with the diameter more preferably less than 1.2 mm and most preferably about 0.85 mm in diameter. The rod has a length of between 4 mm and 15 mm, including the point. The length and diameter are determined such that the desired dose of the formulation can be administered. More preferably the length of the rod is between 4 mm and 10 mm and most preferably between 4 mm and 7 mm in length. The formulation has to be sufficiently robust such that the rod can penetrate the skin without the need for a needle. Typical formulations have a compression strength in excess of 10N as determined by crushing a 4 mm length of the rod on Lloyds Material Tester (LF-Plus), Lloyd Instruments Ltd, Bognor Regis, West Sussex, UK. Preferably the formulations have a compression strength on excess of 15N.

### Stability and Delivery

It was surprisingly found, as described herein, that the therapeutic molecules in the described formulations are unexpectedly stable and retain potency. In some embodiments, after formulation the solubility of the one or more therapeutic molecules at a concentration of at least 50 mg/ml in a physiological solution is greater than 99%. In some embodiments, after formulation the solubility of the one or more therapeutic molecules at a concentration of at least 100 mg/ml in a physiological solution is greater than 99%. A physiological solution, as used herein, preferably is an isotonic solution at neutral pH. Neutral pH, as used herein, includes a pH of about pH7 and a pH of a physiological environment, such as a serum environment.

In some embodiments, the therapeutic molecules are lyophilized prior to use in the formulations described herein. Prior to the instant invention it was not recognized, or expected, that therapeutic molecules comprising immunoglobulin variable domains could be lyophilized and reconstituted without loss of potency.

In one aspect the invention provides high drug load formulations for transdermic delivery of therapeutic molecules comprising immunoglobulin variable domains. In one aspect the invention therefore provides methods for the systemic delivery of immunoglobulin variable domains using needle-free delivery. It was surprisingly found, as described herein, that the invention provides for formulations of 80% by mass or higher of the one or more therapeutic molecules, which does not affect the potency of the one or more therapeutic molecules, such as upon delivery to a subject. For instance, a formulation of 1 mg of at least 80% therapeutic molecule, constructed and arranged as described herein, is capable of delivering to a subject at least 0.8 mg of the one or more therapeutic molecules when administered to a subject. The administration of such a formulation to a subject results in the delivery to the subject of at least 0.8 mg of the one or more therapeutic molecules per administration event.

Potency of the therapeutic molecule can be measured before formulation, after formulation, before administration and/or after administration. For example, the pharmacodynamic and pharmacokinetic properties of the therapeutic molecule can be measured using the methods and assays described in the examples below. In some embodiments, the therapeutic molecule has at least about 90% of the potency after formulation as prior to formulation. In other embodiments, the therapeutic molecule has at least about 90% of the potency after administration as prior to administration. In still other embodiments, the therapeutic molecule has at least about 90% of the potency after administration as prior to formulation. Potency in the formulations described herein can be retained to levels (compared as described herein) of at least about 90%, at least about 91%, at least about 92%, at least about 93%, at least about 94%, at least about 95%, at least about 96%, at least about 97%, at least about 98%, at least about 99%, or even substantially 100% potency.

### Therapeutic Amount

An effective amount of therapeutic is a dosage of the therapeutic molecules comprising the immunoglobulin variable domain (or other protein scaffold) sufficient to provide a medically desirable result. The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. For example, an effective amount for treating a disease or condition would be an amount sufficient to lessen the progression of or inhibit the disease or condition, or its symptoms.

An effective amount of a diagnostic is a dosage of the therapeutic molecules comprising the immunoglobulin variable domain sufficient to provide a medically relevant diagnosis. The effective amount will vary with the particular condition being diagnosed, the age and physical condition of the subject being diagnosed, the severity of the condition, the specific route of administration and like factors within the knowledge and expertise of the health practitioner.

In some embodiments, the formulations and methods of the invention provide per administration a systemic delivery of at least 0.8, at least 0.9, at least 1, at least 1.6, at least 1.8, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 milligrams or more of therapeutic molecules.

### Glide Device

In one aspect, the invention provides formulations and methods for the delivery of therapeutic molecules comprising immunoglobulin variable domains using a Glide Solid Dose Injector (Glide SDI®). The Glide SDI comprises two main components, a single use disposable drug cassette and a reusable actuator. The formulation is preloaded into the drug cassette during manufacture and then packaged to maintain sterility of the formulation. During use the drug cassette, containing the formulation, is placed in the spring powered actuator. The user, which may be a patient, caregiver or healthcare professional, holds the actuator in their hand and pushes the end of the drug cassette against the target tissue. At a preset spring force the actuator triggers and, in doing so, pushes the formulation from the drug cassette into the tissue target. The pointed end of the formulation pierces and penetrates the target tissue without the need for a needle due to the pushing force from the actuator.

### Depot Formulation

In some embodiments, the formulations of the invention are administered by placing the formulations under the skin without the use of a needle-free device. For instance, the formulations can be placed under the skin through injection or surgery. In some embodiments the formulations are solid formulations that are shaped for placement under the skin (e.g., patches, or rods). In some embodiments, the solid formulations of the invention are administered by placing the formulation (such as a rod) under the skin without the use of a needle-free device. For instance, the formulation (such as a rod) can be placed under the skin through injection or surgery.

In some embodiments the formulations are formulations that are prepared for transdermal administration (such as for intravenous, intramuscular or subcutaneous injections), for instance, the formulations can be in suspensions that can be administered through delivery by a needle.

### Needle-Free Delivery Devices

In one aspect the formulations of the invention are delivered to a subject by using a needle-free delivery device. In some embodiments the needle-free delivery device is a Glide Solid Dose Injector (Glide SDI®). Needle-free delivery devices are devices that can administer medicine or diagnostics to a subject by penetrating the skin, but without the use of needle. Non-limiting examples of needle-free delivery devices of the invention are the Glide SDI, jet injectors, powder jet systems, and devices based on iontophoresis or ultrasound transdermal delivery.

The Glide SDI is a simple to use, hand-held, spring powered injection technology. The drug is formulated into a solid dosage form, preferably as a solid rod with a point on one end. The solid formulation is typically produced by mixing the active drug with selected excipients and extruding the material to form a long piece of ‘spaghetti-like’ material. This material is then cut into individual dosages with each formulation have a point on one end and a flat on the other end.

A formulation is loaded into a proprietary packaging known as a drug cassette. The drug cassette is for single use only and comprises a body and a pin. The cassette body provides the structure to hold the formulation and the pin is used to push the formulation from the cassette during the injection process. The length of the pin determines the depth of penetration of the formulation. Typically, the formulation is pushed approximately 1.5 mm beyond the end of the drug cassette meaning that it is injected just under the skin so that the skin closes over the injection site and all of the drug is available for release to the systemic circulation. The formulation can be designed using quick dissolving excipients for immediate release of the active drug to the systemic circulation. Alternatively the formulation can be designed with slower dissolving excipients to enable a controlled or sustained release of the active drug to the systemic circulation over a period of hours, days or even weeks. Alternatively, the formulation can be designed to contain both an immediate release and slow release of one or more active therapeutics in the same formulation. The drug cassette, when it has been filled with the formulation, is packaged to ensure that sterility is maintained. The packaging may also be a barrier for light and moisture to protect the formulation and maintain stability of the formulation.

During use, the drug cassette is removed from the packaging and place in the end of the actuator. The actuator is a hand-held, spring powered device. The user (patient, caregiver or healthcare professional) holds the actuator in their hand and places the end of the drug cassette against the target tissue. The target tissue is likely to be a standard injection site such as the abdomen, upper arm or thigh although other injection sites might be appropriate, especially if local delivery of the formulation is desired.

To actuate the system the actuator is pushed against the skin. The cassette body extends within the actuator and charges the main driving spring within the actuator. At a preset spring force the actuator automatically triggers releasing a striker with the actuator. The striker pushes on the back end of the pin within the cassette which, in turn, pushes the formulation from the drug cassette into the tissue. It is the pointed end of the formulation which creates a small hole in the skin into which the formulation is pushed without the need for a needle. The pin comes to rest on the internal surfaces of the cassette body thus determining the depth to which the formulation is pushed in the skin.

As far as the patient is concerned the formulation is injected instantaneously. The actuator is moved away from the skin and a second spring within the actuator gently starts to push the used cassette from the actuator. The used cassette can then be removed from the actuator and discarded. The second spring automatically resets the actuator so that it is ready for the next injection. The actuator can be used hundreds of times although for certain applications, such as emergency applications, the whole system may be used once and then thrown away.

Although the standard system has just one formulation contained in the drug cassette it is possible that multiple formulations can be injected at the same time.

The standard actuator is designed such that it triggers when the drug cassette is pushed against the skin. This has an inherent safety feature in that the cassette has to be held firmly against the target tissue at the point of actuation. It is also very simple to use because it does not require any coordination. It is also possible to have an actuator in which the spring is primed before it is placed against the skin. The actuator can then be triggered by pressing a button to release the striker.

In some embodiments the needle-free delivery device comprises a powder jet system. Powder jet systems use a supersonic gas flow to deliver a powder medicine or diagnostic. The gas flow accelerates the powder medicine or diagnostic particles to a high speed such that the powder particles can perforate the skin. Powder jet systems are described for instance in U.S. Pat. No. 6,168,587 and Nature Medicine, 6, 1187-1190 (2000).

In some embodiments the needle-free delivery device comprises a delivery device based on iontophoresis. Iontophoresis based delivery involves the interaction between ionized molecules of a medicine or diagnostic and an external electric field, resulting in the migration of charged molecules. Iontophoresis based delivery devices can transport medicine or diagnostic across the skin into the tissues and blood stream (See e.g., U.S. Pat. No. 5,991,655 and U.S. Pat. No. 7,136,698).

In some embodiments the needle-free delivery device comprises based on ultrasound transdermal delivery. Ultrasound delivery devices are described for instance in U.S. Pat. No. 5,814,599 and U.S. Pat. No. 4,767,402.

### Immunoglobulin Variable Domains

In one aspect the invention provides methods, formulations and devices for the needle free delivery of therapeutic molecules comprising immunoglobulin variable domains. In some embodiments the immunoglobulin variable domain is a VH, VL, VHH, camelized VH, camelized VL, or VHH that is optimized for stability, potency, manufacturability and similarity to human framework regions. In some embodiments the immunoglobulin variable domain is a VHH that is optimized for stability, potency, manufacturability and similarity to human framework regions (i.e a humanized or sequence-optimized VHH).

For humanization and sequence-optimization of VHEI's and Nanobodies, reference is generally made to patent applications of Ablynx N.V., for example to WO 2008/020079 and the further prior art cited therein

As for example described on pages 63 and 64 of WO 2008/020079 (incorporated herein by reference), humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from Table A-5 to A-8 on pages 79 to 82 of WO 2008/020079, and/or from a comparision between the sequence of a Nanobody and the sequence of a naturally occurring human V_{H} domain. Similarly, as described on pages pages 63 and 64 of WO 2008/020079 (incorporated herein by reference), camelisation generally involves replacing one or more amino acid residues in the sequence of a naturally occurring V_{H} (such as a human V_{H}) with the amino acid residues that occur at the same position in a V_{HH} domain. Again, suitable camelizing substitutions or combinations of camelizing substitutions will be clear to the skilled person, for example from Table A-5 to A-8 on pages 79 to 82 of WO 2008/020079, and/or from a comparison between the sequence of the V_{H} domain to be camelized and the sequence of a naturally occurring V_{HH} domain.

The humanizing or camelizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The Nanobodies of the invention may be suitably humanized at any framework residue(s), such as at one or more Hallmark residues (as defined herein) or at one or more other framework residues (i.e. non-Hallmark residues) or any suitable combination thereof. One preferred humanizing substitution for Nanobodies of the “P,R,S-103 group” or the “KERE group” is Q108 into L108. Nanobodies of the “GLEW class” may also be humanized by a Q108 into L108 substitution, provided at least one of the other Hallmark residues contains a camelid (camelizing) substitution (as defined herein). For example, as mentioned above, one particularly preferred class of humanized Nanobodies has GLEW or a GLEW-like sequence at positions 44-47; P, R or S (and in particular R) at position 103, and an L at position 108.

The humanized and other analogs, and nucleic acid sequences encoding the same, can be provided in any manner known per se, for example using one or more of the techniques mentioned on pages 103 and 104 of WO 08/020,079.

Also, in addition to humanizing substitutions as described herein, the amino acid sequences of the invention may contain one or more other/further substitutions. Again, some preferred, but non-limiting examples of such other/further substitutions will become clear from the further description herein, and for example may include (and preferably essentially consist of) one or more of the following substitutions:
(a) one or more conservative amino acid substitutions; and/or
(b) one or more substitutions in which a “camelid” amino acid residue at a certain position is replaced by a different “camelid” amino acid residue that occurs at said position, for which reference is for example made to Tables A-5 to A-8 on pages 79 to 82 of 2008/020079, which mention the various Camelid residues that occur as each amino acid position in wild-type VHH's. Such substitutions may even comprise suitable substitutions of an amino acid residue that occurs at a Hallmark position with another amino acid residue that occurring at a Hallmark position in a wild-type VHH (for which reference is for example made to Tables A-5 to A-8 on pages 79 to 82 of 2008/020079); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif.

As mentioned there, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

In some embodiments the therapeutic molecule comprises a multivalent and/or multispecific construct.

Unless indicated otherwise, the term “immunoglobulin variable domain” is used as a general term to include heavy chain variable domain sequences (which are preferred). More specifically, the immunoglobulin variable domains can preferably be heavy chain variable domain sequences that are derived from a conventional four-chain antibody from e.g., human or heavy chain variable domain sequences that are derived from a heavy chain antibody from e.g., camels, dromedaries, alpacas or llamas. According to the invention, the immunoglobulin variable domains can be domain antibodies, or immunoglobulin sequences that are suitable for use as domain antibodies, single domain antibodies, or immunoglobulin sequences that are suitable for use as single domain antibodies or immunoglobulin sequences that are suitable for use as compounds termed “dAbs”, or “Nanobodies” (NANOBODY® and NANOBODIES® are registered trademarks of Ablynx N.V.) in the field and preferably are NANOBODIES® or other immunoglobulin single variable domains. Immunoglobulin variable domains include camelid derived immunoglobulin variable domains and functional, optimized variants thereof. Functional and optimized variants include variants that bind the same epitope and variants that are optimized for better potency in binding, optimized for human framework regions, chemical stability and manufacturability. The immunoglobulin variable domains provided by the invention are preferably in essentially isolated form (as defined herein), or form part of a protein or polypeptide of the invention (as defined herein), which may comprise or essentially consist of one or more immunoglobulin variable domains of the invention and which may optionally further comprise one or more further immunoglobulin variable domains (all optionally linked via one or more suitable linkers). For example, and without limitation, the one or more immunoglobulin variable domains of the invention may be used as a binding unit in such a protein or polypeptide, which may optionally contain one or more further immunoglobulin variable domains that can serve as a binding unit (i.e., against one or more other antigens such as e.g. soluble antigens, membrane antigens, serum proteins such as e.g. serum albumin and/or antigens on the same target in order to result in therapeutic proteins or polypeptides that are multispecific), so as to provide a monovalent, multivalent or multispecific polypeptide of the invention, respectively, all as described herein. Such a protein or polypeptide (or herein also referred to as “Agent of the Invention”) may also be in essentially isolated form (as defined herein).

The invention includes immunoglobulin variable domains of different origin, comprising mouse, rat, rabbit, donkey, shark, human and camelid immunoglobulin variable domains. The invention also includes fully human, humanized or chimeric immunoglobulin variable domains. For example, the invention comprises camelid immunoglobulin variable domains and humanized camelid immunoglobulin variable domains, or camelized domain antibodies, e.g., camelized VH sequences such as described by Hamers et al. (see for example WO 94/04678; Davies and Riechmann (FEES Lett. 339: 285-290, 1994 and Prot. Eng. 9(6): 531-537, 1996); and Muyldermans et al. TIBS 26(4): 230-235, 2001). Moreover, the invention comprises fused immunoglobulin variable domains, e.g., forming a multivalent and/or multispecific construct (e.g., for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin variable domains comprising tags or other functional moieties, e.g. toxins, labels, radiochemicals, etc., which are derivable from the immunoglobulin variable domains of the present invention.

The immunoglobulin variable domain and structure of a immunoglobulin variable domain can—without however being limited thereto—be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. Furthermore, the total number of amino acid residues in an immunoglobulin variable domain can be in the region of 110-120, is preferably 112-115, and is most preferably 113. It should however be noted that parts, fragments, analogs or derivatives (as further described herein) of a immunoglobulin variable domain are not particularly limited as to their length and/or size, as long as such parts, fragments, analogs or derivatives meet the further requirements outlined herein and are also preferably suitable for the purposes described herein.

As used herein, the terms “immunoglobulin variable domains” and “Agents of the Invention” refer to both the nucleic acid sequences coding for the polypeptide and the polypeptide per se. Any more limiting meaning will be apparent from the particular context.

### Protein Scaffolds

It should be appreciated that the formulations, devices and methods for the therapeutic molecules disclosed herein also include polypeptides comprising one or more protein scaffolds. Protein scaffolds, as used herein, includes both antibody-based scaffolds and non-antibody-based scaffolds. Protein scaffolds comprise antigen-binding polypeptides that, in turn, comprise at least one stretch of amino acid residues that correspond to an antibody CDR sequence (i.e., as part of its antigen binding site). Suitable protein scaffolds for presenting antigen-binding sequences will be clear to the skilled person. Non-limiting examples of protein scaffold embraced by the invention are immunoglobulin-based scaffolds, protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), Anticalins, DARPins (designed ankyrin repeat proteins), Adnectins, Chemokine binding proteins identified in ticks, Affilins, CH2 domains, Centyrins (protein fold with significant structural homology to Ig domains with loops analogous to CDRs) and Fynomers (single domain protein scaffolds). See also, Hasse et al. (Protein Science, 2006, 15: 14-27).

Therapeutic Molecule, Multivalent and multispecific Constructs

A therapeutic molecule, as used herein, is any molecule, such as a polypeptide, that comprises one or more immunoglobulin variable domains, including multivalent or multispecific constructs comprising immunoglobulin variable domains. In addition, a therapeutic molecule, as used herein, also includes polypeptides that comprise one or more of the antibody based-scaffolds and non-antibody based scaffolds described herein. It should be appreciated that therapeutic molecules, as used herein, also include diagnostic molecules.

In order to further improve the avidity (i.e., for a desired antigen) of the immunoglobulin variable domains, and/or to provide constructs that can bind to two or more different antigens, two or immunoglobulin variable domains can be combined in a single polypeptide construct, resulting in a multivalent and/or multispecific polypeptide construct. The immunoglobulin variable domains can be coupled to each other directly (e.g., as a fusion protein) or using polypeptide or non-polypeptide linkers. In addition to increasing the binding to the antigen, multiple binding to a target can also increase the therapeutic activity of the multivalent polypeptide relative to the individual immunoglobulin variable domains. Multivalency does not have to be limited to two immunoglobulin variable domains, and as such, multivalent polypeptide constructs can be trimers and tetramers etc. of the same or different immunoglobulin variable domains. In some embodiments immunoglobulin variable domains that bind to different targets are coupled to each other resulting in multispecific polypeptide constructs. In some embodiments one of the immunoglobulin variable domains of the multispecific polypeptide constructs binds a serum protein, thereby increasing the half-life of the polypeptide construct. In some embodiments the serum protein is serum albumin, serum immunoglobulin, thyroxine-binding protein, transferrin or fibrinogen. In some embodiments the multispecific polypeptide constructs also can comprise two or more immunoglobulin variable domains that bind to the same target, thereby increasing the affinity for binding to a single antigen.

Immunoglobulin variable domains (or multivalent and/or multispecific polypeptide constructs) can also be coupled to polypeptides other than antibodies. Coupling of the immunoglobulin variable domains to non-antibody polypeptides can provide the immunoglobulin variable domains with an extra functionality and/or can increase their half-life. Individual immunoglobulin variable domains can be small and can sometimes be disposed of in the body through the kidneys. While immunoglobulin variable domains are more stable than traditional antibodies, their disposal through the kidneys may diminish their therapeutic effectiveness. Filtration by the kidneys can be prevented or reduced by increasing the size of individual immunoglobulin variable domains through multimerization as described above or through coupling to a larger protein, preferably a stable protein found in the bloodstream, like albumin. Coupling immunoglobulin variable domains to larger serum proteins will increase the half-life of the immunoglobulin variable domains. In some embodiments the serum protein is serum albumin, serum immunoglobulin, thyroxine-binding protein, transferrin or fibrinogen. In another embodiment immunoglobulin variable domains are coupled to a polypeptide that would give them additional functionalities. Examples include, but are not limited to, signaling peptides, binding peptides, peptide receptor ligands and functional enzymes.

Immunoglobulin variable domains (or multivalent and/or multispecific polypeptide constructs) can also be coupled to a non-polypeptide group. In one embodiment, the non-polypeptide group is a toxic agent. In another embodiment, the non-polypeptide group is a tracer. The non-polypeptide groups can be coupled to the immunoglobulin variable domain through a linker as is described below.

Coupling the immunoglobulin variable domains to a toxic agent will allow for delivery of the toxin to the antigen. This methodology can be used to introduce a toxic agent to a site where it is most effective (e.g., inside a tumor cell, or on the membrane of a tumor cell). The methodology can also be used to rid the bloodstream of unwanted products. The immunoglobulin variable domains can bind to an unwanted antigen, which can be inactivated by the toxic agent that is attached to the immunoglobulin variable domains. In contrast, if the toxic agent were not attached to a immunoglobulin variable domains, it would not get in close proximity to its target product, or would not stay in sufficiently close proximity long enough, to inactivate the target. Immunoglobulin variable domains can also be coupled to tracers. This will allow for the monitoring of a specific target in the body. For instance, a immunoglobulin variable domains that binds to a tumor antigen can be coupled to a radioactive tracer. The amount of radioactivity retained in the body and the localization of the tracer will help diagnose the amount of tumor cells in the body and can help determine the progress of a specific treatment regimen (See also below).

Amino acid linkers for use in multivalent and multispecific polypeptides will be clear to the skilled person, and for example include Gly-Ser linkers, for example of the type (GlyₓSer_{y})_{z}, such as for example (Gly₄Ser)₃ or (Gly₃Ser₂)₃, as described in WO 99/42077, hinge-like regions such as the hinge regions of naturally occurring heavy chain antibodies or similar sequences. Linkers can also provide some functionality for the multivalent or multispecific polypeptides. For example, linkers containing one or more charged amino acid residues can provide improved hydrophilic properties, whereas linkers that form or contain small epitopes or tags can be used for the purposes of detection, identification and/or purification.

Immunoglobulin variable domains can be connected to each other, to other polypeptides or to non-polypeptides groups through a number of non-peptide linkers. In one embodiment, the linker comprises an amido linker moiety, an amino linker moiety, a carbonyl linker moiety, a carbamate linker moiety, a urea linker moiety, an ether linker moiety, a disulphide linker moiety, a succinamidyl linker moiety, a succinyl linker moiety, and combinations thereof. In other embodiments, the linker moiety is an ester including: carbonate (—OC(O)O—), succinoyl, phosphate esters (—O—(O)POH—O—), sulfonate esters, and combinations thereof.

### Diagnostics

In one embodiment, needle-free delivery devices are used to administer immunoglobulin variable domains used for diagnostics. Coupling a tracer to a immunoglobulin variable domain will allow for the determination of an amount and/or location of a specific antigen in vivo or in vitro. The tracer can be, but is not limited to an agent of fluorescent or radioactive origin. The diagnostic administered with the needle-free delivery device can be used to determine the amount and/or location of a variety of antigens, for instance a solid tumor cell marker or peptide marker in the bloodstream. In most diagnostic assays, the diagnostics needs to be administered a set time prior to the assay. While the readout of the diagnostic assay will likely need to be performed by a health care official, being able to self administer the diagnostic dose will allow for one less trip to the clinic and savings in time and cost. Immunoglobulin variable domains are preferred because of their stability and small size. They could for instance enter a tumor cell, allowing for a more complete diagnostic picture.

### Diseases and Disorders

A variety of diseases and disorders can be treated using immunoglobulin variable domains as delivered according to the methods, formulations and devices of the invention. Exemplary diseases include inflammatory disorders, aggregation-mediated disorders, cancers, autoimmune diseases, neurodegenerative disorders, genetic disorders,

“Inflammatory disorders” include diseases such as rheumatoid arthritis, Crohn's disease, mastocytosis, asthmas, multiple sclerosis, inflammatory bowel syndrome and allergic rhinitis (see, e.g., US published application 2006/0058340). For example, in one embodiment, the needle-free delivery device is used to administer (polypeptides comprising one or more) immunoglobulin variable domains against TNF-alpha, against IL-6, against RANK-L, against IL-6R, or against any other protein or target involved in the IL-6 pathway, as for example described in WO 2004/041862, US 2005/0054001, US 2006/0034845, U.S. 60/682,332, WO 03/050531, WO 03/054016, U.S. 60/782,243, U.S. 60/782,246, WO 06/122786, WO 04/003019 and WO 03/002609.

For the prevention and treatment of aggregation-mediated disorders and/or thrombotic disorders, for example, immunoglobulin variable domains against vWF (as for example described in WO 2004/062551, U.S. Ser. No. 10/541,708, U.S. 60/683,474 and WO 06/122825) may be formulated as and administered using the needle-free delivery devices disclosed herein.

A variety of cancers can be treated according to the methods, formulations and device of the invention. The cancer may be a carcinoma or a sarcoma but it is not so limited. For example, the cancer may be basal cell carcinoma, biliary tract cancer, bladder cancer, bone cancer, brain cancer, breast cancer, cervical cancer, choriocarcinoma, CNS cancer, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, acute lymphoid leukemia, acute myeloid leukemia, chronic lymphoid leukemia, chronic myeloid leukemia, cutaneous T-cell leukemia, hairy cell leukemia, liver cancer, non-small cell lung cancer, small cell lung cancer, lymphoma, follicular lymphoma, Hodgkin's lymphoma, Non-Hodgkin's lymphoma, melanoma, myeloma, multiple myeloma, neuroblastoma, oral cavity cancer, ovarian cancer, pancreatic cancer, prostate cancer, rectal cancer, renal cancer, cancer of the respiratory system, retinoblastoma, rhabdomyosarcoma, skin cancer, squamous cell carcinoma, stomach cancer, testicular cancer, thyroid cancer, cancer of the urinary system and uterine cancer (US published application 2006/0019923).

An “immune disorder” includes adult respiratory distress syndrome, arteriosclerosis, asthma, atherosclerosis, cholecystitis, cirrhosis, Crohn's disease, diabetes mellitus, emphysema, hypereosinophilia, inflammation, irritable bowel syndrome, multiple sclerosis, myasthenia gravis, myocardial or pericardial inflammation, osteoarthritis, osteoporosis, pancreatitis, rheumatoid arthritis, scleroderma, and ulcerative colitis (see, e.g., US published application 2003/0175754).

“Neurodegenerative disorder” or “neurodegenerative disease” or “neuropathology” refers to a wide range of diseases and/or disorders of the central and peripheral nervous system, such as Parkinson's disease, Alzheimer's disease (AD), amyotrophic lateral sclerosis (ALS), denervation atrophy, otosclerosis, stroke, dementia, multiple sclerosis, Huntington's disease, encephalopathy associated with acquired immunodeficiency disease (AIDS), and other diseases associated with neuronal cell toxicity and cell death (see, e.g., US published application 2006/0025337). For example, for the prevention and/or treatment of Alzheimer's disease, polypeptides comprising one or more) immunoglobulin variable domains against amyloid-beta (as for example described in U.S. 60/718,617 and WO 06/040153) may be formulated as and administered using the needle-free delivery devices disclosed herein.

“Genetic disorders” include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, SRY: sex determination, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum or Zellweger syndrome. (see, e.g., 2005/0281781).

In addition, molecules that can be targeted (“targets”) by the immunoglobulin variable domains delivered according to the methods of the invention include, for example, targets of foreign origin, host derived cellular targets, and host derived non-cellular targets. Exemplary targets are listed below.

Foreign target agents include drugs, especially drugs subject to abuse such as heroin and other opiates, PCP, barbiturates, cocaine and derivatives thereof, benzodiazepines, etc., poisons, toxins such as heavy metals like mercury and lead, chemotherapeutic agents, paracetamol, digoxin, free radicals, arsenic, bacterial toxins such as LPS and other gram negative toxins, Staphylococcus Toxins, Toxin A, Tetanus toxins, Diphtheria toxin and Pertussis toxins, plant and marine toxins, virulence factors, such as aerobactins, radioactive compounds or pathogenic microbes or fragments thereof, including infectious viruses, such as hepatitis B, A, C, E and delta, CMV, HSV (type 1, 2 & 6), EBV, varicella zoster virus (VZV), HIV-1, -2 and other retroviruses, adenovirus, rotavirus, influenzae, rhinovirus, parvovirus, rubella, measles, polio, reovirus, orthomyxovirus, paramyxovirus, papovavirus, poxvirus and picornavirus, prions, protists such as plasmodia tissue factor, toxoplasma, filaria, kala-azar, bilharziose, entamoeba histolitica and giardia, and bacteria, particularly gram-negative bacteria responsible for sepsis and nosocomial infections such as E. coli, Acynetobacter, Pseudomonas, Proteus and Klebsiella, but also gram positive bacteria such as staphylococcus, streptococcus, etc. Meningococcus and Mycobacteria, Chlamydiae, Legionnella and Anaerobes, fungi such as Candida, Pneumocystis carini, and Aspergillus, and Mycoplasma such as Hominis and Ureaplasma urealyticum (see, e.g., U.S. Pat. No. 5,843,440).

Host derived cellular and non-cellular targets against to which the immunoglobulin variable domains and constructs that are administered may be directed will be clear to the skilled person and for example include, but are not limited to, all targets for which NANOBODIES®, dAb's or other immunoglobulin variable domains (or polypeptides comprising the same) have been proposed in the art (such as TNF-alpha, Von Willebrand factor, interleukins such as IL-6, amyloid-beta, etc., as well as the other targets mentioned in the prior art referred to herein), as well as more generally cellular and non-cellular targets for which antibodies or antibody fragments (including but not-limited to ScFv constructs) have been proposed in the art.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

### Lyophilisation of Nanobody ALX0081sv1 (vWF0002)

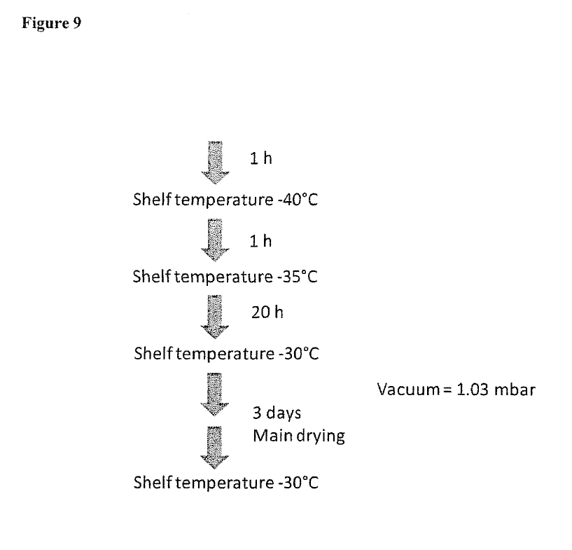

Glass vials with 5.45 ml solution containing 150 mg vWF0002 (SEQ ID NO: 1) diluted in Milli-Q H₂O+0.02% Tween 80 are frozen at −70° C. The frozen lyophilisation vials with the vWF0002 samples are then put on the shelf of the lyophilizer which was pre-cooled to −40° C. The scheme for the lyophilisation cycle is indicated in FIG. 9. When the lyophilisation process is completed, the pressure inside the lyophilizer is increased to 200 mbar before closing the vials and opening the lyophilizer to remove the vials. Samples are closed with a crimped cap and stored at −20° C.

The sequence of vWF0002 is SEQ ID NO: 1
DVQLVESGGGLVQPGGSLRLSCAASGRTFSYNPMGWFRQAPGKGRELVAA
ISRTGGSTYYPESVEGRFTISRDNAKRTVYLQMNSLRAEDTAVYYCAAAG
VRAEQGRVRTLPSEYTFWGQGTQVTVSSAAAEVQLVESGGGLVQPGGSLR
LSCAASGRTFSYNPMGWFRQAPGKGRELVAAISRTGGSTYYPESVEGRFT
ISRDNAKRTVYLQMNSLRAEDTAVYYCAAAGVRAEQGRVRTLPSEYTFWG
QGTQVTVSS

### Example 2

### Lyophilisation of vWF0002 does not Result in Aggregation of the Compound

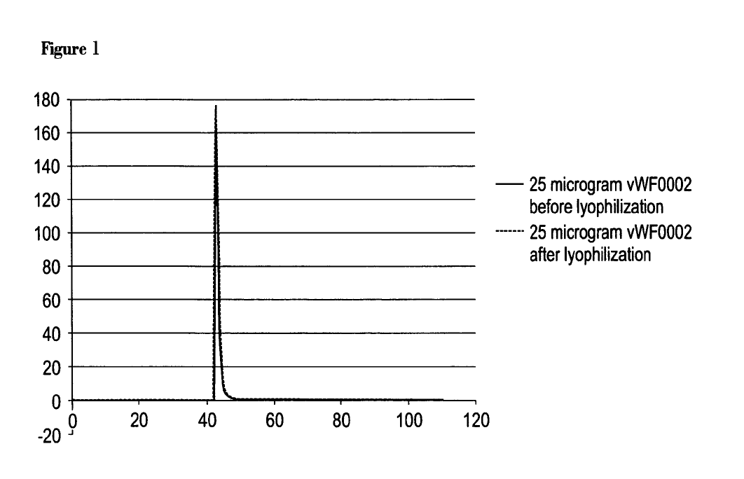

After the lyophilisation process, one vial of lyophilised vWF0002 is resuspended in 5.45 ml saline solution (0.9% NaCl) and analyzed by size exclusion chromatography (SEC) to check if the lyophilisation process has induced aggregates in the sample. The SEC analyses are performed on an Agilent 1200 series HPLC on a Phenomenex BioSep SEC S 2000 column. FIG. 1 shows there is no difference in the SEC profiles the re-dissolved lyophilised vWF0002 protein and the control vWF0002 sample and no aggregates are detected.

### Example 3

### Lyophilisation has No Impact on the Homogeneity of vWF0002

vWF0002 redissolved after lyophilisation as described in example 1 is analyzed with reverse phase chromatography (RPC) to check if lyophilisation affects homogeneity of the compound. The analyses are performed on an Agilent 1200 series HPLC on a Zorbax 300SB C3 column using an acetonitrile-TFA gradient.

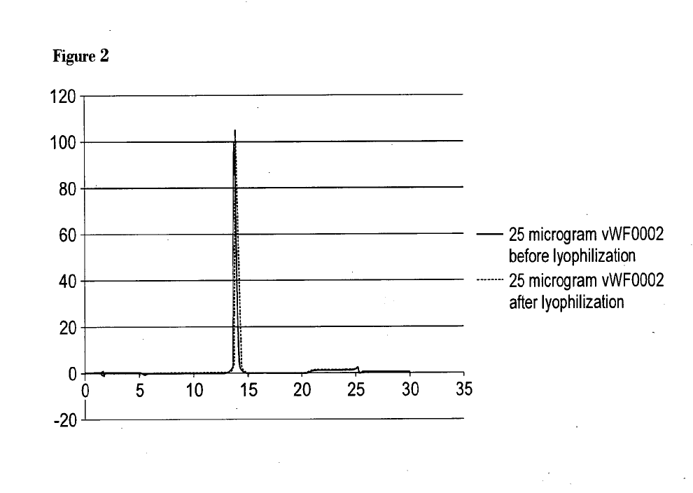

The RPC data (FIG. 2) show that resuspended lyophilised vWF0002 Nanobody is completely homogeneous and that there are no differences between the control sample (before lyophilisation) and the lyophilised sample supporting the observation that the lyophilisation does not seem to influence the folding of the Nanobody, nor does it induce the formation of chemical variants such as oxidation or deamidation which would appear as discrete peaks separated from the main peak.

### Example 4

### Manufacture of Nanobody Formulations

A set of formulations was manufactured to incorporate the maximum amount of Nanobody vWF0002 in a 10 mg weight rod. To this end, sodium CarboxyMethyl Cellulose (CMC), Dextran and MethylCellulose (MC) are used as the principal binders. These parenterally acceptable polymeric excipients are chosen since they can be incorporated at relatively low levels to impart sufficient strength.

The extrusion process involves mixing of the Nanobody vWF0002 compound and the excipients, adding the granulation fluid, producing a dry paste and extruding the paste. A first formulation was prepared to contain 80% of Nanobody vWF0002 and 20% of Dextran. A second formulation was prepared to contain 90% of Nanobody and 10% of sodium carboxymethyl cellulose. A third formulation was prepared to contain 80% of Nanobody vWF0002 and 20% methylcellulose. After extrusion, the paste is dried by overnight storage at 2-8° C. in a dessicator.

### Example 5

### Physico-Mechanical Strength, Disintegration and Penetration of a Nanobody vWF0002 Formulation Containing 20% Methylcellulose

To obtain a 10 mg weight rod of a formulation to contain 80% of Nanobody vWF0002 and 20% methylcellulose, the length of the rod was determined to be approximately 11 mm when the formulation has been extruded through a die with an orifice diameter of 1.1 mm. The length of a 14 mg formulation was determined to be approximately 13.5 mm.

Table A shows the physico-mechanical properties of the formulation in addition to disintegration data and penetration results in vitro. The three point bend and compression tests are undertaken on a Lloyds Material Testing machine (LF-Plus 2156). Disintegration tests are by visual assessment of the formulation when dropped into water for injection at both room temperature and at 37° C. The penetration success is a binary result in-vitro in a silicon test-bed (Polymer System Technology—R31-2186) using the Glide SDI with the device configuration used in Example 8 for a pharmacokinetic study.

TABLE A

| Average weight uniformity | 9.97 mg | (n = 22) |
|---|---|---|
| Three point bend on dosage unit (N) | 8.32 ± 4.10 | (n = 5) |
| Compression on dosage unit (N) | 12.98 ± 2.50 | (n = 5) |
| Compression on 2.5 mm length (N) | 30.38 ± 4.72 | (n = 4) |
| Disintegration at Room Temperature | 2277 ± 283 | (n = 5) |
| on dosage unit (s) | | |
| Disintegration at 37° C. on dosage | >6300 | (n = 2) |
| unit (s) - incubator | | |
| Penetration success (in-vitro) % | 100% | (n = 5) |

### Example 6

### Extrusion of the Different Formulations of Lyophilised vWF0002 into Rods does not Affect the Quality of vWF0002 Nanobody

The different formulations of the lyophilised vWF0002 that were extruded into rods are analyzed and compared with the lyophilised vWF0002 that was not extruded into rods after reconstitution into isotonic salt solution. Three formulations are analyzed incorporating 90% vWF0002 and 10% sodium-carboxymethyl cellulose (CMC) as excipient, 80% vWF0002 and 20% Dextran, or 80% vWF0002 and 20% methylcellulose (MC) respectively. The total weight of the rods was 10 mg, a small portion of lyophilised compound or a piece of rod was taken and weighed on an analytical balance. Physiological saline (NaCl 0.9%) was added to achieve a final concentration of 10 mg/ml Nanobody. Samples were slightly turbid upon re-dissolution in the saline. Samples were centrifuged and diluted 1/10 before the Optical Density (OD) measurements at 280 and 320 nm.

TABLE 1
Recovery of vWF0002 after lyophilisation.

| | | | | | Expected | |
|---|---|---|---|---|---|---|
| | | | | Concentration | concentration | |
| | Material | OD280 | OD320 | in mg/mL° | mg/mL | % recovery |
| Batch 1 | vWF0002 | 1.229 | 0.004 | 8.3 | 10.0 | 83 |
| | lyophilised | | | | | |
| Batch 1 | Rod 90/10 | 1.140 | 0.006 | 7.7 | 9.0 | 86 |
| | CMC | | | | | |
| Batch 1 | Rod 80/20 | 1.096 | 0.007 | 7.4 | 8.0 | 93 |
| | Dextran | | | | | |
| Batch 2 | Rod 90/10 | 1.158 | 0.004 | 0.78 | 9.0 | 87 |
| | CMC | | | | | |
| Batch 2 | Rod 80/20 | 1.015 | 0.005 | 0.68 | 8.0 | 85 |
| | Dextran | | | | | |
| Batch 2 | Rod 80/20 | 1.037 | 0.003 | 0.69 | 8.0 | 86 |
| | MC | | | | | |

°using the following formula: ((OD280 − OD320)/1.48) * 10 where 1.48 is the absorbance coefficient for a solution of 1 mg/mL vWF0002 10 is the dilution factor

As shown in Table 1, recoveries of material after lyophilisation is in this experiment 83% which is somewhat lower than other lyophilisation experiments performed in similar conditions where on general recoveries ranging from 90-95% were obtained (data not shown). Recoveries for the rods are acceptable and not significantly different to that of the reconstitution after lyophilisation.

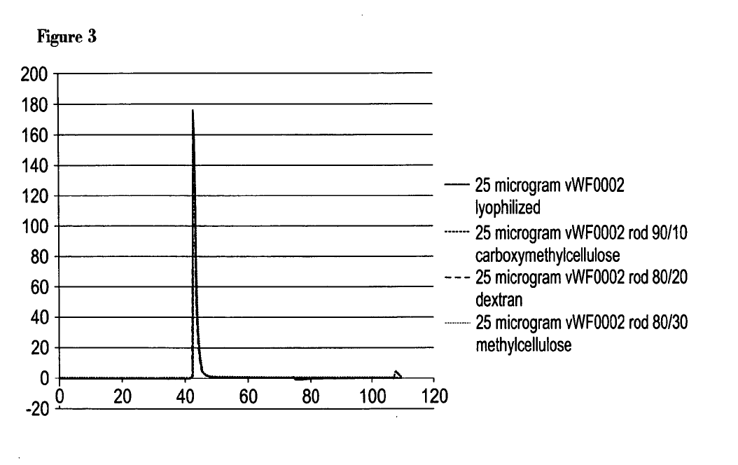

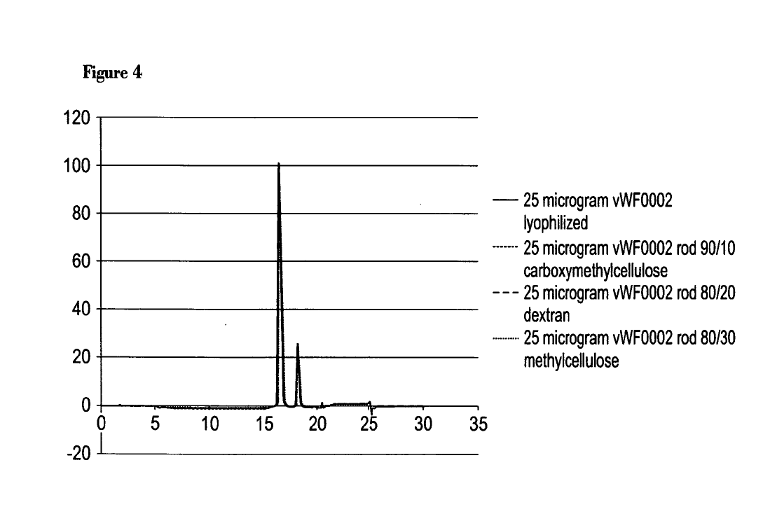

The resuspended lyophilised vWF0002 and the formulations 90/10 CMC, 80/20 Dextran and 80/20 MC are analyzed with size exclusion chromatography (SEC) and reverse phase chromatography (RPC) as described above. As shown in FIG. 3, there is no difference in the SEC profiles of the different formulated vWF0002 and the re-dissolved lyophilised vWF0002 compound. Likewise, a complete overlay of the RPC profiles of the different formulated vWF0002 and the re-dissolved lyophilised vWF0002 compound is observed (FIG. 4). Taken together, these data show that formulation of Nanobody vWF0002 into rods incorporating 80-90% of Nanobody and 10-20% of CMC, Dextran or MC as excipient, has no influence on the homogeneity of the Nanobody vWF0002. The Nanobody recovered after reconstitution show identical profiles to the material before and after lyophilisation and demonstrate that the material is still monomeric after reconstitution (i.e., no soluble aggregates can be detected in the SEC profile, while the recoveries by OD suggest there is no significant protein loss due to insoluble aggregates). Moreover, the RPC profiles show that the reconstituted material did not undergo any major chemical and/or structural change; i.e., any oxidation of the vWF0002 would appear as a pre-peak on RPC, while badly folded materials would be retarded in their elution behavior. No such phenomena were observed.

In FIG. 4 there is a second peak present in the RPC chromatogram at about +2 min retention time from the main peak. This peak is a product related variant of the vWF0002 which lacks one of the building blocks the formation of the canonical disulfide bond. This does not affect the potency of the material (data not shown) and was shown to be absent in other batches.

The potency of Nanobody vWF0002 before and after formulation into rods was analyzed by surface plasmon resonance. vWF A1-domain was immobilized on a CM5 sensor chip surface docked in Biacore 3000 by amine coupling using NHS/EDC for activation and ethanolamine for deactivation. Approximately 1600RU of vWF A1-domain was immobilized. Next, the chip is preconditioned by injecting 100 ng/ml vWF0002 10 times. All samples were diluted to 100 ng/ml, 50 ng/ml and 25 ng/ml (in triplicate) in running buffer (HBS-EP+0.5 M NaCl) and analyzed on the chip (triplicates crossed). The samples were injected for 2 min at a flow rate of 45 ul/ruin over the activated and reference surfaces. Evaluation was done using BIAevaluation software. The data from the fitted data were transferred into excel. Parallel line analysis was calculated with the PLA 1.2 software to determine parallelism between reference and analyte. The potency of the materials is expressed versus the potency of the reference material stored at −70° C. (non lyophilised). Values are expressed as relative potency versus this reference. During the experimental analysis sufficient controls are included to determine the stability of the chip.

The relative potency of the lyophilized vWF0002 is 94.8% to that of the reference; a relative potency of 94.6 or 96.1% is observed for a 3 mg vWF0002 rod 80/20 MC (two independent analyses). For the vWF0002 reconstituted from a 90/10 CMC rod, a relative potency of 117% is measured and 93.3% for vWF0002 reconstituted from a 80/20 Dextran rod. Taken together, these surface Plasmon resonance analyses show no significant loss of biological activity of vWF0002 when formulated into rods.

### Example 7

### Use of Needle-Free Delivery Glide SDI Device to Deliver 3 mg vWF0002 80/20 MC Rods During Preclinical PK and Efficacy Study

Glide SDI is a needle-free drug delivery system for the injection of drugs and vaccines in solid doses. The drug formulation is pushed into the skin using the simple spring powered Glide SDI actuator. The Glide drug cassette contains the drug in a solid dosage form. The active drug is mixed with excipients, as required, to produce a robust solid formulation that can be injected into the skin. The drug component is formulated and prefilled into a drug cassette. The drug cassette is a single use component and contains no sharps, so it can be safely thrown away with normal household waste following use. The aim of the study was to assess the pharmacokinetics, bioavailability and local tolerance of vWF0002, hereafter termed test item, in pigs after a single subcutaneous administration or after subcutaneous implantation of 3 mg vWF0002 rod 80/20 MC solid dosage formulation using the Glide SDI device.

### Experimental Set-Up

German landrace pigs were used as non-rodent species for the pharmacokinetic study and showed cross-reactivity with the administered test item. In addition, the skin morphology of pigs closely resembles that of human skin.

Each treatment group consisted of 3 animals and the animals were allocated to the test groups employing a pseudo-random body weight stratification procedure that yielded groups with approximately equal mean body weight. The administration and dose levels were as described in Table 2.

TABLE 2
description of administration and
dose levels of test item vWF0002.

| | | Dose level | | Animal |
|---|---|---|---|---|
| | Administration | [μg/kg body | Administration | reference |
| Group | route | weight] | volume [μL/kg] | no. |
| 1 | s.c. injection | 80 | 53 | 1-3 f |
| 2 | s.c. implantation | 80 | A single 3 mg | 4-6 f |
| | of a solid dosage | | solid dosage | |
| | formulation using | | form/animal | |
| | the Glide SDI | | | |
| | technology | | | |

Group 1 received a single subcutaneous bolus injection under the skin in the inguinal region (right side). Group 2 received a single subcutaneous implantation of a 3 mg solid dosage formulation under the skin in the inguinal region (right side) using the Glide SDI device. The test item (solution) for subcutaneous administration was provided as a ready-to-use injectable with a concentration of 1.5 mg/ml. Upon thawing the material, the tube was thoroughly mixed by inverting the tube several times to make sure the material was homogenized. The solid dosage form of the test item for subcutaneous implantation was provided as a ready-to-use.

Following administration, the implantation site of the treated groups was clearly marked and a photo of the injection sites was made immediately.

### Local and Systemic Tolerance:

To score for local tolerance, the injection site was assessed 1 h prior and after the injection of the test item and once daily for all other days of the in-life study. The reactions were scored as follows (based on DRAIZE, Appraisal of the Safety of Chemicals in Food, Drugs and Cosmetics, Association of Food, Drug Officials of the United States, Austin, Tex., 1959).

| Erythema and eschar formation | Value |
|---|---|
| No erythema | 0 |
| Very slight erythema (barely perceptible) | 1 |
| Well-defined erythema | 2 |
| Moderate to severe erythema | 3 |
| Severe erythema (beef redness) or eschar formation (injuries in | 4 |
| depth) preventing erythema reading | |

| Oedema formation | Value |
|---|---|
| No oedema | 0 |
| Very slight oedema (barely perceptible) | 1 |
| Slight oedema (edges of area well defined | 2 |
| by definite raising) | |
| Moderate oedema (raised approx. 1 millimetre) | 3 |
| Severe oedema (raised more than 1 millimetre | 4 |
| and extending beyond area of exposure) | |

No signs of local intolerance reactions were noted for any of the animals after a single subcutaneous administration at a dose level of 80 μg/kg body weight, or after a subcutaneous implantation of the 3 mg solid dose formulation. Furthermore, no erythema and oedema formations were noted at the injections sites. No histopathology was examined, as no injection site reactions had been observed.

No test-item related signs of systemic intolerance reactions were noted for any of the animals after a single subcutaneous administration at a dose level of 80 μg/kg body weight, or after a subcutaneous implantation of the 3 mg solid dose formulation.

Also, no influence on the body weight was noted for any of the animals after a single subcutaneous administration at a dose level of 80 μg/kg body weight, or after a subcutaneous implantation of the 3 mg solid dose formulation (data not shown).

### Blood Sampling for Pharmacokinetics and Pharmacodynamics:

Sufficient whole blood was withdrawn from the vena cava craniales of all animals in order to obtain the required volume Na-Citrate^{plasma/animal/} sampling time.

| | Aliquot |
|---|---|
| Sampling times | volume |
| 0 (pre-dose) | 2 × 1 mL |
| Test day 1: 30 min, 1, 2, 3, 4, 6, 8 and 12 h p.a. | 2 × 1 mL |
| Test day 2: 24 h p.a. | |
| Test day 3: 48 h p.a. | |
| Test day 4: 72 h p.a. | |
| Test day 5: 96 h p.a. | |
| Test day 8: 168 h p.a. | 2 × 1 mL |

Total number of aliquots:

The samples were cooled immediately using an IsoTherm-Rack system (Eppendorf AG, 22331 Hamburg, Germany) until centrifugation (within 30 min of blood withdrawal). Blood was mixed well and centrifuged for 15 min at 2200×g at room temperature. The upper layer (PPP, platelet poor plasma) was transferred to a fresh tube and immediately stored at −80° C. or colder until shipment for analysis.

### Pharmacokinetic Profiles

The pharmacokinetic profile of the test item after a subcutaneous implantation of the 3 to mg solid dose formulation was analyzed in female Landrace pigs and was compared to the test item after a single subcutaneous administration at a dose level of 80 μg/kg body weight

For bioanalytical determination of the test items in pig plasma, plasma samples were tested for levels of the test item after a single subcutaneous administration at a dose level of 80 μg/kg body weight, or after a subcutaneous implantation of the 3 mg solid dose formulation using ELISA based PK assays. The detection of test item in the ELISA assays is based on the binding of these Nanobodies with vWF and the assay set-ups are as such that total vWF-binding Nanobody is detected.

Briefly, 96-well microtiter plates (Maxisorp, Nunc, Wiesbaden, Germany) were coated overnight at 4° C. with neutravidin (Pierce) in 10:10 buffer at 3 μg/ml. Wells were aspirated and blocked for 1 hour at RT with PBS/1% casein. After this blocking step, wells were washed with PBS/0.05% Tween20. A biotinylated bivalent Nanobody against test item was added to the neutravidin coated plate at 2 μg/ml in PBS/0.1% casein and incubated for 1 hour at RT. After the incubation step of this capture tool, wells were washed 3 times with PBS/0.05% Tween20.

Preparations of the standards, QC samples and dilutions of the test samples were performed in a non-coated (polypropylene) plate. For the standard curve and QC-samples, solutions at the required concentrations were prepared in PBS/0.1% casein and spiked into 100% pig plasma. To prepare standards and QC samples, a 1/100 dilution of the pure pig plasma dilutions was made in IgM-Reducing Agent (Immunochemistry Technologies, Bloomington, USA) supplemented with 2.5% pooled human plasma (referred to as sample diluent). For the test samples, dilution factors for the test samples were estimated. Samples were diluted 1/100 in sample diluent in a first step, and if needed, further dilution was done in sample diluent supplemented with 1% pig plasma.

Standards, QC samples and diluted test samples were transferred onto the coated plate and incubated for 1 hour at RT. Afterwards the plates were washed followed by a complexation step with purified vWF (ZLB Behring) diluted in PBS/0.1% casein for 30 minutes at RT. Plates were washed and Nanobody/vWF complexes bound to the capture tool detected with Rabbit anti-human vWF Ab (Dako, Denmark), diluted 1/2000 in PBS/0.1% casein and incubated for 30 minutes at RT. After washing, a 1/15,000 dilution in PBS/0.1% casein of Horse-Radish-Peroxidase labelled goat anti-rabbit Ab (Dako, Denmark) was added to the plate and incubated for 30 minutes at RT. The enzyme coupled to the Ab catalyzes a chemical reaction with the substrate sTMB (3,3′,5,5′-tetramethylbenzidine, SDT reagents, Brussels, Belgium), resulting in a colorimetric change. After stopping this reaction after 10 minutes using HCl (IN), the intensity of the colour was measured using a spectrophotometer at 450 nm.

The concentrations of the test items in the plasma samples were determined based on the parameters of a 4-parameter logistic fit of the standard curve. All test samples were tested in 2 independent runs and the reported values are the average of the 2 analysis batches.

For pharmacokinetic data analysis, descriptive statistics (mean and SD) were calculated per dose group and per sampling time point using Microsoft Excel 2007. In case all three values were BQL, BQL was reported. When one or two out of three values were BQL, BQL values were set to zero and the mean calculated. Individual plasma concentration-time profiles were subjected to non-compartmental analysis (NCA) using WinNonlin Pro 5.1 (Pharsight Corporation, USA; 2006). The area under the curve (AUC) was estimated using the lin up/log down rule. LLOQ values were treated as missing, except when comprised between two values above the LLOQ, then they were set to zero. The concentration at time zero (CO) was estimated through back-calculation based on the two first data points. The terminal elimination half-life (t1/2) was calculated automatically (best-fit) using a log-linear regression of the non-zero concentration-time data of the log-linear portion of the terminal phase. A minimum of three points were considered for the determination of λz.

The following main pharmacokinetic parameters were estimated: the plasma concentration at time zero (CO); the area under the plasma concentration-time curve extrapolated to infinity (AUCinf), total body clearance (CL), volume of distribution at steady-state (Vdss), and the dominant half-life (t_{1/2}, dominant), and the terminal half-life (t½).

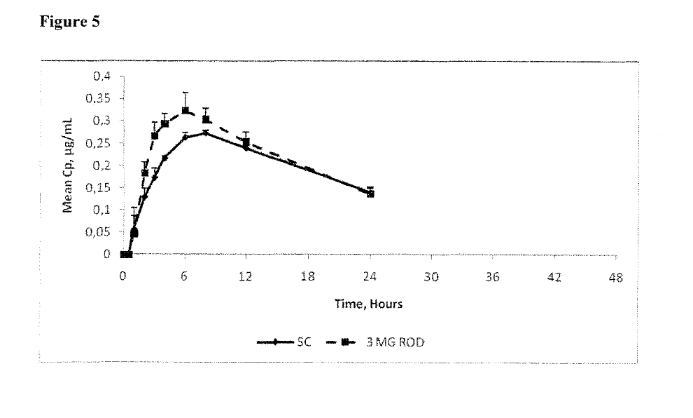

in FIG. 5, the mean plasma concentration time profiles of the test item vWF0002 after a subcutaneous implantation of the 3 mg solid dose formulation and after a single subcutaneous administration at a dose level of 80 μg/kg body weight are shown. Subcutaneous implantation of the 3 mg solid rod formulation of vWF0002 results in a similar mean plasma concentration time profile compared with a single subcutaneous administration of vWF0002 at a dose level of 80 μg/kg body weight. However, subcutaneous implantation of the 3 mg slid rod formulation of vWF0002 shows a somewhat faster Cmax compared to the subcutaneous administration at a dose level of 80 μg/kg b.w, as depicted in FIG. 5 and Table 3. Dose normalized Cmax and AUC after the subcutaneous implantation of the 3 mg solid rod formulation of vWF0002 was comparable to the Cmax obtained after subcutaneous administration at a dose level of 80 μg/kg body weight. Cmax was attained at a slightly faster time (6 h) for the 3 mg vWF0002 solid rod formulation relative to the subcutaneous administration of vWF002 at the equivalent dose level of 80 μg/kg body weight (7.3 h). Finally, a comparable t½ was obtained for vWF0002 when administered after the subcutaneous implantation of the 3 mg solid rod formulation or after subcutaneous administration at an equivalent dose level of 80 μg/kg body weight.

TABLE 3
Pharmacokinetic parameters of vWF0002 after a subcutaneous
implantation of a 3 mg 80/20 MC solid dosage formulation at a
dose level of 80 μg/kg body weight and after a single subcutaneous
administration at a dose level of 80 μg/kg body weight.

| | Formulation of | | | | | |
|---|---|---|---|---|---|---|
| | vWF0002 | | | | | |
| | SC | | | 3 mg rod SC | | |
| | Dose | | | | | |
| | 80 μg/kg | | | 80 μg/kg | | |
| | Mean | SD | % CV | Mean | SD | % CV |
| Cmax (μg/mL) | 0.27 | 0.006 | 2.11 | 0.32 | 0.04 | 12.5 |
| Tmax (h) | 7.33 | 1.15 | 15.7 | 6 | 0 | 0 |
| T½ (h) | 16.3 | 1.2 | 7.34 | 13.8 | 0.96 | 6.98 |
| AUCinf (μg * h/mL) | 8.085 | 0.69 | 8.57 | 8.063 | 0.86 | 10.7 |
| DN Cmax (μg/mL) | 0.27 | | | 0.32 | | |
| DN AUCinf | 8.085 | | | 8.063 | | |
| (μh * h/mL) | | | | | | |
| Frel (%) | | | | 100% | | |

From Table 3 it is also appreciated that the relative bioavailability of vWF0002 from the 3 mg solid rod formulation is 100% when compared to the subcutaneous administered vWF0002 as a liquid solution at an equivalent dose level of 80 μg/kg body weight, and demonstrated full dissolution of the test item in vivo.

### Pharmacodynamic Profile and Activity Assays

Pharmacodynamic characteristics upon compound administration were measured by means of a ristocetin cofactor activity assay (Biopool). The ristocetin cofactor activity is a functional assay for VWF, measuring the capacity of VWF to interact with the platelet receptor glycoprotein Ib using ristocetin as a modulator.

For pharmacodynamic data analysis, descriptive statistics (mean and SD) were calculated per dose group and per sampling time point using Microsoft Excel 2007. Response parameters and associated statistics for the overall time course were calculated by noncompartmental analysis of the response-time data using WinNonlin Pro 5.1 (Pharsight Corporation, USA; 2006). The non-compartmental analysis was based on a model for pharmacodynamic data (Model 220). The threshold value was set at 20% based on extensive historical PK/PD data on the vWF0002 compound. The following main pharmacodynamic parameters were determined: time below the threshold (Time below T), area under the threshold (AUC below T), time at which the % RICO first drops below the threshold (tₒₙₛₑₜ), and time at which the % RICO first returns back above the threshold (t_{offset}).

For determining the inhibition of ristocetin-induced binding of VWF to Platelets, microtiter plates (Maxisorp, Nunc) were coated overnight at 4° C. with 0.1 mg/mL poly-L-Lysine (Sigma, St Louis, Mo.) in PBS. After 3 times washing with phosphate buffered saline (PBS), wells were incubated for 1 hour at room temperature (RT) with formalin fixed human platelets (Dade Behring, Newark, Del.) which were diluted two-fold in PBS or—as a blank—with PBS. Wells were washed 3 times with PBS and blocked for 2 hours at RT with PBS containing 4% bovine serum albumin (BSA, Sigma). A dilution series of test item was prepared in human plasma and was preincubated for 30 min at RT with 1.5 mg/mL ristocetin (ABP, NJ, USA) after which the mixture was transferred to the coated wells. After 1.5 hours incubation at 37° C., wells were washed 6 times with PBS and residual bound vWF was detected for 1 hour at RT with a 1/2000 dilution of anti-VWF polyclonal antibodies labeled with horse radish peroxidase (Dako, Glostrup, Denmark). Visualization was obtained with esTIvIB (SDT reagents, Germany) and the coloring reaction was stopped with 1M hydrochloric acid after which the absorbance was determined at 450 nm. For the analysis of the data, the absorbance values were corrected using the absorbance of the respective blanks.

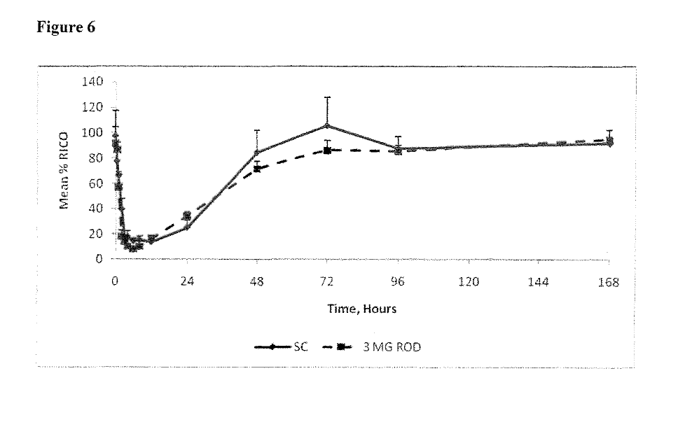

In FIG. 6, the temporal time profiles of the % RICO measurements following a single subcutaneous administration at a dose level of 80 μg/kg body weight, or after a subcutaneous implantation of the 3 mg solid dose formulation respectively in pig are shown. The results confirm the in vivo activity of vWF0002 in the pig and demonstrate that test item after a single subcutaneous administration at a dose level of 80 μg/kg body weight, or after a subcutaneous implantation as a 3 mg solid dose formulation completely block the ristocetin-induced binding of VWF to the platelet surface. Time below the threshold (20% RICO) is slightly longer when test item is administered as a single subcutaneous administration at a dose level of 80 μg/kg body weight compared with subcutaneous implantation as a 3 mg solid dose formulation.

Table 4 depicts the basic pharmacodynamic parameters and demonstrated that the mean Rmin is somewhat lower with the 3 mg solid rod formulation compared with a single subcutaneous administration of test item vWF0002 at an equivalent dose level of 80 μg/kg body weight (6.61% versus 14%). The mean time of maximum effect (Tmin) was earlier after administration of a 3 mg solid rod formulation compared with a single subcutaneous administration of test item vWF0002 at an equivalent dose level of 80 μg/kg body weight (4.33 versus 8.00 h). The mean AUC-below-T is slightly higher after administration of the 3 mg solid rod formulation compared with a single subcutaneous administration of test item vWF0002 at an equivalent dose level of 80 μg/kg body weight (88.2% RICO*h versus 64.4% RICO*h). Finally, the mean time-below-T was slightly longer for test item vWF0002 when administered as a subcutaneous soluble formulation compared with the administration of a 3 mg solid rod formulation (15.9 versus 12.1 h).

TABLE 4
Basic pharmacodynamic parameters for vWF0002 after a subcutaneous
implantation of a 3 mg 80/20 MC solid dosage formulation at a dose level
of 80 μg/kg body weight and after a single subcutaneous administration
at a dose level of 80 μg/kg body weight.

| | Formulation | | | | | |
|---|---|---|---|---|---|---|
| | SC | | | 3 mg rod SC | | |
| | Dose | | | | | |
| | 80 μg/kg | | | 80 μg/kg | | |
| | Mean | SD | % CV | Mean | SD | % CV |
| Rmin (%) | 14.0 | 1.88 | 13.4 | 6.61 | 3.13 | 47.4 |
| Tmin (h) | 8.00 | 3.46 | 43.3 | 4.33 | 1.53 | 35.3 |
| AUC_below_T | 64.4 | 39.0 | 60.6 | 88.2 | 38.2 | 43.3 |
| (% * h) | | | | | | |
| Time_below_T (h) | 15.9 | 4.08 | 25.7 | 12.1 | 1.97 | 16.3 |

### Example 8

### Use of Needle-Free Delivery Glide SDI Device to Deliver 10 mg vWF0002 80/20 MC Rods During Preclinical PK and Efficacy Study

In this study the pharmacokinetics, bioavailability and local tolerance of vWF0002 in pigs was assessed after subcutaneous implantation of a 10 mg rod 80/20 MC solid dosage formulation using the Glide SDI device. Three female Landrace pigs received a single subcutaneous implantation of a 10 mg vWF0002 solid dosage formulation under the skin in the inguinal region (right side) using the Glide SDI device.

Local and systemic tolerance was assessed as described in example 6; no signs of local intolerance reactions were noted for any of the animals after subcutaneous implantation of the 10 mg solid dose formulation, reaching a concentration of 276 μg vWF0002/kg body weight. Furthermore, no erythema and oedema formations were noted at the injections sites. No histopathology was examined, as no injection site reactions had been observed. No test-item related signs of systemic intolerance reactions were noted for any of the animals after subcutaneous implantation of the 10 mg solid dose formulation.

Also, no influence on the body weight was noted for any of the animals after subcutaneous implantation of the 10 mg solid dose formulation (data not shown).

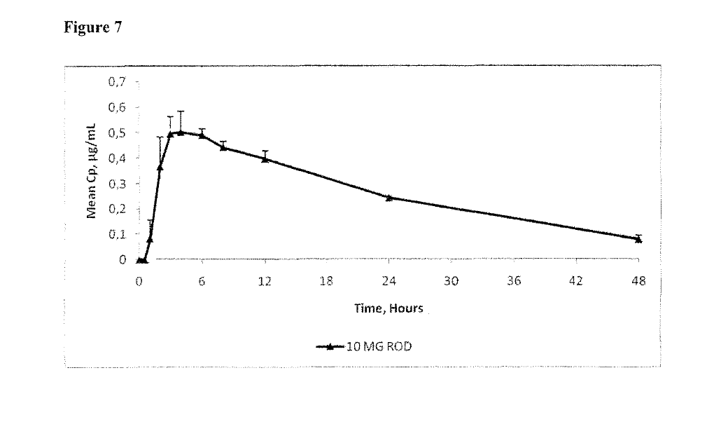

The following main pharmacokinetic parameters were estimated: the plasma concentration at time zero (C0); the area under the plasma concentration-time curve extrapolated to infinity (AUCinf), total body clearance (CL), volume of distribution at steady-state (Vdss), and the dominant half-life (t_{1/2}, dominant), and the terminal half-life (t½). In FIG. 7, the mean plasma concentration time profile of the test item vWF0002 after a subcutaneous implantation of the 10 mg solid dose formulation, reaching a dose level of 276 μg/kg body weight, is shown. Table 5 depicts the main pharmacokinetic parameters and shows that Cmax is attained after 4.3 h. The AUC reaches an average of 14.69 μg*h/ml and a t½ of 16.8 h is obtained.

TABLE 5
Pharmacokinetic parameters of vWF0002 after subcutaneous
implantation of a 10 mg 80/20 MC solid dosage formulation
at a dose level of 276 μg/kg body weight.
Formulation 10 mg rod SC
Dose 276 μg/kg

| | Mean | SD | % CV |
|---|---|---|---|
| Cmax (μg/mL) | 0.53 | 0.055 | 10.5 |
| Tmax (h) | 4.33 | 1.53 | 35.3 |
| T½ (h) | 16.8 | 3.02 | 18 |
| AUCinf (μg*h/mL) | 14.69 | 0.23 | 1.59 |

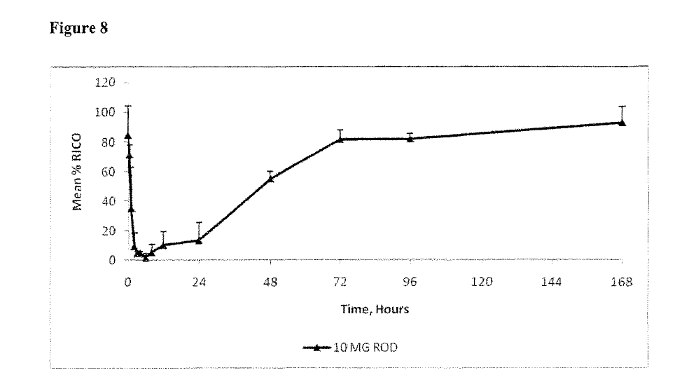

The temporal time profiles of the % RICO measurements following subcutaneous implantation of the 10 mg solid dose formulation in pig are shown in FIG. 8. The results confirm the in vivo activity of vWF0002 in the pig and demonstrate that test item after subcutaneous implantation as a 10 mg solid dose formulation completely blocks the ristocetin-induced binding of VWF to the platelet surface. Time below the threshold lasted to about 27-28 h. Table 6 depicts the basic pharmacodynamic parameters and shows that a pronounced mean maximum effect (Rmin) of 1.67% was reached. The mean time of maximum effect (Tmin) was reached at 3.67 h post-administration. The mean AUC-below-T and mean time-below-T were 278% RICO*h and 24.3 h respectively. The rapid onset of action (as evaluated by the % RICO measurements) and prolonged time below the threshold (24.3 h) and mean AUC under the threshold (278% RICO*h) observed for subcutaneous implantation of a vWF0002 10 mg rod 80/20 MC solid dosage formulation using the Glide SDI device, corresponds well with the rapid Cmax of 4.3 h.

TABLE 6
Pharmacodynamic parameters of vWF0002 after subcutaneous
implantation of a 10 mg 80/20 MC solid dosage formulation
at a dose level of 276 μg/kg body weight.
Formulation 10 mg rod SC
Dose 276 μg/kg

| | Mean | SD | % CV |
|---|---|---|---|
| Rmin (%) | 1.67 | 2.9 | 173.7 |
| Tmin (h) | 3.67 | 2.08 | 56.7 |
| AUC_below_T (%*h) | 278 | 224 | 80.6 |
| Time_below_T (h) | 24.3 | 7.87 | 32.4 |

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety, particularly for the use or subject matter referenced herein.

1. A formulation, comprising:
one or more therapeutic molecules and one or more excipients,
wherein the formulation comprises at least 80% by mass of the one or more therapeutic molecules, and
wherein the one or more therapeutic molecules comprise one or more immunoglobulin variable domains.

2. The formulation of claim 1, wherein the formulation comprises at least 90% by mass of the one or more therapeutic molecules.

3. The formulation of claim 1, wherein the formulation is a solid.

4. The formulation of claim 1, wherein the formulation is shaped for delivery to a subject through the subject's skin.

5. The formulation of claim 1, wherein the formulation is a rod formulation.

6. The formulation of claim 1, wherein the solubility of the one or more therapeutic molecules at a concentration of at least 50 mg/ml in a physiological solution is greater than 99%.

7. The formulation of claim 1, wherein the solubility of the one or more therapeutic molecules at a concentration of at least 100 mg/ml in a physiological solution is greater than 99%.

8. The formulation of claim 6, wherein the physiological solution is isotonic and has a neutral pH.

9. The formulation of claim 1, wherein the formulation is at least about 1 milligram in total mass.

10. The formulation of claim 9, wherein the formulation is at least about 3 milligrams in total mass.

11. The formulation of claim 10, wherein the formulation is at least about 10 milligrams in total mass.

12. The formulation of claim 11, wherein the formulation is at least about 14 milligrams in total mass.

13. The formulation of claim 9, wherein administration of the formulation to a subject results in the delivery to the subject of at least 0.8 mg of the one or more therapeutic molecules per administration event.

14. The formulation of claim 9, wherein the formulation is constructed and arranged to deliver to a subject at least 0.8 mg of the one or more therapeutic molecules when administered to a subject.

15. The formulation of claim 10, wherein administration of the formulation to a subject results in the delivery to the subject of at least 2.4 mg of the one or more therapeutic molecules per administration event.

16. The formulation of claim 10, wherein the formulation is constructed and arranged to deliver to a subject at least 2.4 mg of the one or more therapeutic molecules when administered to a subject.

17. The formulation of claim 11, wherein administration of the formulation to a subject results in the delivery to the subject of at least 8 mg of the one or more therapeutic molecules per administration event.

18. The formulation of claim 11, wherein the formulation is constructed and arranged to deliver to a subject at least 8 mg of the one or more therapeutic molecules when administered to a subject.

19. The formulation of claim 1, wherein the immunoglobulin variable domain is a VH, VL, VHH, camelized VH, camelized VL, or VHH that is optimized for stability, potency, manufacturability and similarity to human framework regions.

20. The formulation of claim 1, wherein the immunoglobulin variable domain is a VHH that is optimized for stability, potency, manufacturability and similarity to human framework regions.

21. The formulation of claim 1, wherein the therapeutic molecule comprises a multivalent and/or multispecific construct.

22. A needle-free delivery device comprising the formulation of claim 1.

23. The needle-free delivery device of claim 22, wherein the needle-free delivery device comprises:
i) a housing;
ii) a force generator configured to generate a force capable of pushing the formulation from a packaging into a human or animal body;
iii) a force transmitter configured to transmit said force to push the formulation from the packaging into the human or animal body; and,
iv) a triggering element configured to trigger the device.

24. A method for administering to a subject the formulation of claim 1 comprising:
administering to the subject the formulation of claim 1 by using a needle-free delivery device.

25. The method of claim 24, wherein the needle-free delivery device comprises:
i) a housing;
ii) a force generator configured to generate a force capable of pushing the formulation from a packaging into a human or animal body;
iii) a force transmitter configured to transmit said force to push the formulation from the packaging into the human or animal body; and,
iv) a triggering element configured to trigger the device.

26. The method of claim 24, wherein the one or more therapeutic molecules has at least about 90% of the potency after formulation as prior to formulation.

27. The method of claim 24, wherein the one or more therapeutic molecules has at least about 90% of the potency after administration as prior to administration.

28. The method of claim 24, wherein the one or more therapeutic molecules has at least about 90% of the potency after administration as prior to formulation.

29. The method of claim 26, wherein the potency is at least about 95%.

30. The method of claim 29, wherein the potency is at least about 99%.


SEQUENCE LISTING (1 sequence)

SEQ ID NO: 1 - protein, 259 aa, Artificial Sequence
  Nanobody construct
DVQLVESGGGLVQPGGSLRLSCAASGRTFSYNPMGWFRQAPGKGRELVAAISRTGGSTYYPESVEGRFTISRDNAKRTVYLQMNSLRAEDTAVYYCAAAGVRAEQGRVRTLPSEYTFWGQGTQVTVSSAAAEVQLVESGGGLVQPGGSLRLSCAASGRTFSYNPMGWFRQAPGKGRELVAAISRTGGSTYYPESVEGRFTISRDNAKRTVYLQMNSLRAEDTAVYYCAAAGVRAEQGRVRTLPSEYTFWGQGTQVTVSS